SEQUENCE LISTING (4 sequences)

SEQ ID NO: 1 - DNA, 21 bp, Unknown
  Synthetic Oligonucleotide Containing AP-1 Recognition Sequence
cgcttgatgagtcagccggaa

SEQ ID NO: 2 - DNA, 21 bp, Unknown
  Synthetic Oligonucleotide Containing AP-1 Recognition Sequence
gcgaactactcagtcggcctt

SEQ ID NO: 3 - DNA, 22 bp, Unknown
  Synthetic Oligonucleotide Containing SP-1 Recognition Sequence
attcgatcggggcggggcgagc

SEQ ID NO: 4 - DNA, 22 bp, Unknown
  Synthetic Oligonucleotide Containing SP-1 Recognition Sequence
taagctagccccgccccgctcg

(19) United States
(12) Patent Application Publication
(10) Pub. No.: US 2004/0198800 A1
(43) Pub. Date: Oct. 7, 2004

(54) Lipoxygenase inhibitors as hypolipidemic and anti-hypertensive agents

(76) Inventors: Geoffrey Allan, Richmond, VA (US); Glen Kelley, Glen Allen, VA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/734,625

(22) Filed: Dec. 15, 2003

Related U.S. Application Data
(60) Provisional application No. 60/434,412, filed on Dec. 19, 2002.
(60) Provisional application No. 60/511,103, filed on Oct. 15, 2003.

(51) Int. Cl. A61K 31/415; A61K 31/192

(52) U.S. Cl. 514/406; 514/575; 514/570

(57) ABSTRACT

The present invention provides a method for treating elevated serum triglycerides or hypertension via administering to a human subject with elevated serum triglycerides or hypertension an effective amount of pharmaceutical composition comprising a 5-lipoxygenase inhibitor, in an effective amount which is sufficient to reduce elevated serum triglycerides or hypertension, wherein the 5-lipoxygenase inhibitor is not NDGA or curcumin.

This application claims benefit of priority to U.S. Provisional Application No. 60/434,412, filed Dec. 19, 2002 and to U.S. Provisional Application No. 60/511,103, filed Oct. 15, 2003, which applications are hereby incorporated by reference in their entirety.

### BACKGROUND OF THE INVENTION

1. Field of the Invention

The present invention provides pharmaceutical methods to reduce human hyperlipidemia, elevated serum triglycerides, and/or hypertension by administering lipoxygenase (LO) inhibiting compounds.

2. Related Art

NDGA (nordihydroguaiaretic acid or CAS # 500-38-9) is a compound that exhibits multiple biological properties including lipoxygenase inhibition and tyrosine kinase inhibition. This compound has demonstrated hypolipidemic and antihypertensive properties in in vivo animal models (Maya et al., Am. J. Physiol. Endocrinol. Metab., 279: E593-600 (2000); Scribner et al., Metabolism, 49:9 1106-1110 (2000)). The hypolipidemic and antihypertensive properties are not ascribed to the lipoxygenase properties of NDGA. In fact, the authors of this study suggest that some other biological property may be responsible for these biological effects.

NDGA has the structure as follows:

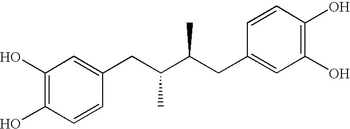

Curcumin (CAS # 458-37-7) is a compound that exhibits multiple biological properties including lipoxygenase inhibition. This compound has demonstrated hypolidemic properties in an in vivo diabetic rat model, although no basis for the bioactivity is described. (Babu et al., Mol. Cell. Biochem., 166 (1-2): 169-175 (1997)).

Curcumin has the structure as follows:

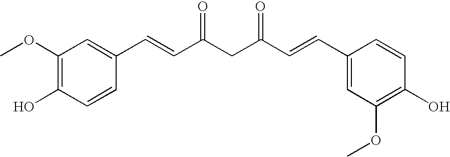

Both NDGA and curcumin are poorly bioavailable, and thus must be administered in prohibitive (and possibly toxic) concentrations.

Nadler et al. (U.S. Pat. No. 6,191,169) describe the role of 12-LO in the pathogenesis of diseases including atherosclerosis, breast cancer, autoimmune, inflammatory disease, diabetic vascular and kidney disease and insulin resistance. This patent does not describe hypolipidemic or anti-hypertensive properties of lipoxygenase inhibitors.

Das (Exp. Biol. Med., 227(11): 989-997 2002) raises the hypothesis of a relationship between metabolic syndrome and chronic low grade inflammation. There are numerous reports that relate the inflammatory cytokine TNF alpha to obesity and diabetes.

Han et al. (Diabetes Care, 25(11): 2016-2021 (2002)) and others demonstrate a relationship between inflammatory markers and diabetes and/or diabetic complications, and cardiovascular disease.

Furthermore, high fructose feeding causes diet-induced alterations of lipid metabolism and decreased insulin sensitivity with alterations of hepatic pyruvate dehydrogenase (Park et al., Biochem. J, 282: 753-757 (1992)) and hepatic VLDL secretion (Zavaroni et al., Metabolism, 31:1077-1083 (1982)). Inflammatory cytokines also induce dramatic changes in lipid metabolism, particularly in serum triglycerides via increased hepatic secretion and/or delayed clearance of VLDL (Feingold et al., Z. Ernhrungswiss 37:66-74 (1998)).

Since the introduction of high fructose corn sweeteners in 1967, the amount of fructose consumption has steadily risen and now accounts for about 9% of daily caloric intake in the United States. Unlike glucose, which is widely utilized by tissues throughout the body, fructose is primarily metabolized in the liver (Hallfrisch et al., FASEB J., 4: 2652-2660 (1990); Bantle et al., Am. J. Clin. Nutr., 72: 1128-1134 (2000)).

High fructose fed (HFF) diets induce well characterized metabolic dysfunction, typically resulting in a rapid elevation of serum triglycerides with a corresponding increase in blood pressure within two weeks. Animals maintained on this diet for longer periods of time develop elevated free fatty acids and hyperinsulinemia at the expense of glycemic control. In this metabolic model, compounds that lower circulating lipid levels, increase insulin sensitivity, or inhibit TNFα production reduce serum triglycerides and improve blood pressure (Inoue et al., Metabolism, 44: 1626-1630 (1995); Mangaloglu et al., Metabolism, 51: 409-418 (2000)). Moreover, if animals are subjected to an exercise regimen, the diet-induced effects can be ameliorated (Zavaroni et al., Metabolism, 31:1077-1083 (1982)). Thus this animal model exhibits many of the hallmarks of an early stage of the Metabolic Syndrome (or “Syndrome X”), in which a combination of physical inactivity and diet results in cardiovascular disease and metabolic complications.

Therefore, the use of certain anti-inflammatory compounds may prove useful in the treatment of metabolic syndrome, diabetes, or related metabolic diseases. Such compounds may also be useful to prevent the exacerbation of disease or prevent development of complications. There are numerous anti-inflammatory “targets” in the art. The art does not teach the use of 5-lipoxygenase inhibitors as a specific class of anti-inflammatory agents that would be useful in the treatment of hyperlipidemia or hypertension. In addition, there has been a longstanding problem with the in vivo use of 5-lipoxygenase inhibitors due to poor oral bioavailabilty for most of these compounds (Bhattacherjee et al., Ann. New York Acad. Sci., 307-320 (1988)). Due to the chronic nature of lipid lowering and hypertensive therapeutic regimens, there is a strong need for the compound to be administered by a convenient route, preferably orally, in order to ensure ease of use and patient compliance.

There is therefore, a need for new means to control serum lipids and control hypertension using orally available anti-inflammatory agents. This need and others are provided in the present invention that describes methods of treating a subject suffering from hyperlipidemia comprising administration of a therapeutically effective amount of a 5-lipoxygenase inhibitor.

### SUMMARY OF THE INVENTION

The present invention provides a method for treating elevated serum triglycerides or hypertension comprising administering to a human subject with elevated serum triglycerides or hypertension an effective amount of a pharmaceutical composition comprising a 5-lipoxgenase inhibitor, said amount being sufficient to reduce said elevated serum triglycerides, wherein said 5-lipoxygenase inhibitor is not NDGA or curcumin.

The present invention provides a method for treating elevated serum triglycerides or hypertension comprising administering to a human subject with elevated serum triglycerides or hypertension an effective amount of a pharmaceutical composition comprising a 5-lipoxygenase inhibitor selected from the group consisting of an acetohydroxamic acid derivative, a phenyl pyrazoline derivative, a 2-(12-hydroxydodeca-5,10-diynyl)-3,5,6-trimethyl-1,4-benzoquinone derivative, and a 3-[1-(4-chlorobenzyl)-3-t-butyl-thio-5-isopropylindol-2-yl]-2,2-dimethyl propanoic acid derivative.

The present invention also provides a method for treating elevated serum triglycerides or hypertension comprising administering to a human subject with elevated serum triglycerides or hypertension an effective amount of pharmaceutical composition comprising 4,5-dihydro-1-(3-(trifluoromethyl)phenyl)-1H-pyrazol-3-amine (BW 755c), said amount being sufficient to reduce said elevated serum triglycerides.

The present invention further provides a method for treating elevated serum triglycerides or hypertension comprising administering to a human subject with elevated serum triglycerides or hypertension an effective amount of pharmaceutical composition comprising N-(3-phenoxycinnamyl)acetohydroxamic acid (BW 4AC), said amount being sufficient to reduce said elevated serum triglycerides.

In addition, the present invention provides a method for treating elevated serum triglycerides or hypertension comprising administering to a human subject with elevated serum triglycerides or hypertension an effective amount of pharmaceutical composition comprising 2-(12-Hydroxydodeca-5,10-diynyl)-3,5,6-trimethyl-1,4-benzoquinone (AA861), said amount being sufficient to reduce said elevated serum triglycerides.

In addition, the present invention provides a method for treating elevated serum triglycerides or hypertension comprising administering to a human subject with elevated serum triglycerides or hypertension an effective amount of pharmaceutical composition comprising 3-[1-(4-chlorobenzyl)-3-t-butyl-thio-5-isopropylindol-2-yl]-2,2-dimethylpropanoic acid (MK 886), said amount being sufficient to reduce said elevated serum triglycerides.

### BRIEF DESCRIPTION OF THE FIGURES

FIG. 1 shows the effect of two 5-LO inhibitor compounds on serum triglycerides in a diet-induced model of hypertriglyceridemia and hypertension.

FIG. 2A is a scatter graph of animal body weights before and after oral administration of 5-lipoxygenase inhibitors in a diet-induced model of hypertriglyceridemia and hypertension.

FIG. 2B is a scatter graph of free fatty acids (FFA) and serum triglycerides (TG) before and after oral administration of 5-lipoxygenase inhibitors in a diet-induced model of hypertriglyceridemia and hypertension.

FIG. 2C is a scatter graph of serum glucose and insulin before and after oral administration of 5-lipoxygenase inhibitors in a diet-induced model of hypertriglyceridemia and hypertension.

FIG. 3 represents the animal treatment paradigm of Example 2.

FIGS. 4A-4E show the hepatic lipid composition of chow and HFF diet fed animals. Chow diet=clear bars; HFF diet=dotted bars; HFF diet+NDGA treatment=stripped bars; HFF+BW 755c treatment=hatched bars.

FIGS. 5A-5C show western blot analyses of kinase activity in chow and HFF diet fed animals. Chow diet=clear bars; HFF diet=dotted bars.

FIGS. 6A-6C show quantified EMSA analysis of hepatic AP-1 and SP-1 in chow and HFF diet fed animals. Chow diet=clear bars; HFF diet=dotted bars; HFF diet+NDGA treatment=stripped bars; HFF+BW 755c treatment=hatched bars.

FIGS. 7A-7B show the serum corticosterone measurement in chow and HFF diet fed animals. Chow diet=clear bars; HFF diet=dotted bars; HFF diet+NDGA treatment=stripped bars; HFF diet+BW 755c treatment=hatched bars.

FIG. 8 is a schematic of the hepatic metabolism of fructose. F=fructose; FIP=fructose-1-phosphate; DHAP=dihydroxyacetonephosphate; TG=triglyceride; G3P=glyceraldehyde-3-phosphate; MG=methylglyoxal AGE=advanced glycation end product intermediate.

### DETAILED DESCRIPTION OF THE INVENTION

Definitions

The term “5-lipoxygenase inhibitor” as used herein refers to compounds that interfere with the pathway of the metabolism of arachidonic acid. The lipoxygenases are a family of enzymes that catalyze the oxygenation of arachidonic acid. The enzyme 5-lipoxygenase converts arachidonic acid to 5-hydroperoxyeicosatetraenoic acid (5-HPETE). This is the first step in the metabolic pathway yielding 5-hydroxyeicosatetraenoic acid (5-HETE) and the important class of mediators, the leukotrienes (LTs). Such compounds may inhibit enzyme activity through a variety of mechanisms. By the way of example, the inhibitor may block or reverse the association of the enzyme with the membrane or inhibit the translocation of specific enzymes such as 5-lipoxygenase via a protein such as 5-lipoxygenase-activating protein (FLAP). Alternatively, the inhibitors used in the methods described herein may block the enzyme activity directly by acting as a substrate for the enzyme or by depriving the enzyme of necessary cofactors.

The term “elevated serum triglycerides” refers to a serum triglyceride level above the normal range and at a level that may pose health risks to the individual. In humans triglycerides are considered “elevated” if the total serum triglyceride level is greater than 150 mg/dL.

The term “concurrently” as used herein refers to either administration of a single composition comprising both active agents or administration of individual compositions comprising the two active agent administered in a time frame over which the subject receives the benefit of the combination of both active agents. As such, the subject could receive a 5-lipoxygenase inhibitor and then an anti-diabetic compound, a lipid-lowering medication or an anti-hypertensive compound or vice versa.

The term “lipid lowering medication” refers to HMG-CoA reductase inhibitors, compounds that are inhibitors of 3-hydroxy-3-methylglutaryl-CoA reductase. Compounds that have inhibitory activity for HMG-CoA reductase can be readily identified by using assays well-known in the art. For example, see the assays described or cited in U.S. Pat. No. 4,231,938 and PCT Publication No. WO 84/02131. Examples of HMG-CoA reductase inhibitors that may be used include, but are not limited to, lovastatin (NIEVACORO; see U.S. Pat. Nos. 4,231,938; 4,294,926; 4,319,039), simvastatin (ZOCORO; see U.S. Pat. Nos. 4,444,784; 4,820,850; 4,916,239), pravastatin (PRAVACHOLO; see U.S. Pat. Nos. 4,346,227; 4,537,859; 4,410,629; 5,030,447; 5,180,589), fluvastatin (LESCOLO; see U.S. Pat. Nos. 5,354,772; 4,911,165; 4,929,437; 5,189,164; 5,118,853; 5,290,946; 5,356,896), atorvastatin (LIPITORO; see U.S. Pat. Nos. 5,273,995; 4,681,893; 5,489,691; 5,342,952) and cerivastatin (also known as nivastatin and BAYCHOLO; see U.S. Pat. No. 5,177,080). The term HMG-CoA reductase inhibitor as used herein includes all pharmaceutically acceptable lactone and open-acid forms (i.e., where the lactone ring is opened to form the free acid), as well as salt and ester forms of compounds which have HMG-CoA reductase inhibitory activity. Therefor, the use of such salts, esters, open-acid and lactone forms is included within the scope of this invention.

Abbreviations are as follows: HFF (high fructose-fed diet); TG (triglyceride); NDGA (nordihydroguaiaretic acid); BW 755c (4,5-Dihydro-1-(3-(trifluoromethyl)phenyl)-1H-pyrazol-3-amine); LO (lipoxygenase).

The methods of the present invention are directed to the use of 5-lipoxygenase inhibitors or derivatives thereof in the prevention and treatment of hyperlipidemia or hypertension. There are numerous 5-lipoxygenase inhibitors that are well known in the art, and method to make and test said compounds are well known.

In one embodiment, the 5-lipoxygenase inhibitor is a acetohydroxamic acid derivative such as N-(3-phenoxycinnamyl)acetohydroxamic acid (See U.S. Pat. No. 4,738,986). This compound is also known as BW 4AC or CAS# 106328-57-8.

BW 4AC as used herein has the formula:

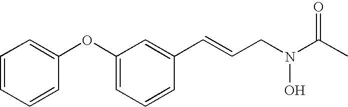

In one embodiment, the 5-lipoxygenase inhibitor is a phenyl pyrazoline derivative, such as 4,5-dihydro-1-(3-(trifluoromethyl)phenyl)-1H-pyrazol-3-amine. (Radmark et al., FEBS Lett., 110: 213 (1980)). This compound is also known as BW 755c or CAS#-66000-40-6.

BW 755c as used herein has the formula:

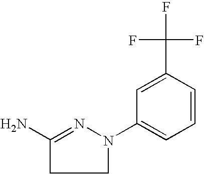

In one embodiment, the 5-lipoxygenase inhibitor is 2-(12-Hydroxydodeca-5,10-diynyl)-3,5,6-trimethyl-1,4-benzoquinone (AA861) or derivatives thereof (Yoshimoto et al, Biochemical Biophysica ACTA, 713: 470-473 (1982); Ashida et al., Prostoglandins, 26(6): 955 (1993)). AA861 was disclosed in U.S. Pat. No. 4,393,075.

In one embodiment, the 5-lipoxygenase inhibitor is 3-[1-(4-chlorobenzyl)-3-t-butyl-thio-5-isopropylindol-2-yl]-2,2-dimethylpropanoic acid (MK886), also identified as CAS 118414-82-7, or derivatives thereof. (Gillard et al., Can. J. Physiol. Pharmacol., 67: 456-464 (1989); Rouzer et al., Journal of Biological Chemistry, 265: 1436-1442 (1990); U.S. Pat. No. 5,081,138). Derivatives of MK886 are also intended to be encompassed by this invention. Derivatives of MK886 intended to be encompassed by this invention include, but are not limited to, L-669,572 (3-[1-(p-cholorobenzyl)-5-isopropyl-3-cyclo-propylmethylthioindole-2-yl]-2,2-dimethylpropanoic acid); L-663,511 (3-[1-(p-cholorobenzyl)-5-isopropyl-3-phenysulfonylindol-2-yl)-2,2-dimethylpropanoic acid); L-665,210 (3-[1-(p-chlorobenzyl)-5-isopropyl-3-phenysulfonylindol-2-yl)-2,2-dimethylpropanoic acid); L-654-639 (3[1-(p-chlorobenzyl)-5-methoxy-3-methylindol-2-yl]-2,2-dimethylpropanoic acid); and L-668,017 (See Rouzer et al., Journal of Biological Chemistry, 265: 1436-1442 (1990)), which is herein incorporated by reference. In a preferred embodiment, the MK886 derivative is 3-(1-(4-chlorobenzyl)-3-(1-butyl-thio)-5-(quinolin-2-yl-methoxy)-indol-2-yl)-2,2-dimethyl propanoic acid) (MK-591) (Tagari et al., Agents Action, 40:62-71 (1993)).

MK886 as used herein has the formula:

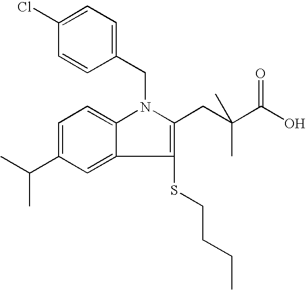

If desired, the effective daily dose of the active ingredients may be administered as one, two, three, four, five, six or more sub-doses administered separately at appropriate intervals throughout the day, optionally, in unit dosage forms.

Combination Therapy

The present invention also provides methods of treating hyperlipidemia and hypertension comprising administration of a 5-lipoxygenase inhibitor and at least a second compound. The second compound is an anti-diabetic, lipid lowering medication, or an anti-hypertensive compound. Anti-diabetic compounds include metformin, sulfonylureas, PPAR agonists, and the like. Lipid lowering compounds include HMG-CoA inhibitors and bezafibrates. Preferably said combination therapy is conducted where the 5-lipoxygenase inhibitor and the second compound are administered as a concurrent regimen.

We hypothesized that high fructose fed (HFF) animals exhibited altered lipid metabolism due to hepatic stress due to the burden of fructose metabolism. Additionally, fructose bypasses two regulatory steps of glycolysis, glucokinase and phosphofructokinase, thus potentially providing unregulated accumulation of glycolytic intermediates. Thus, studies were conducted to determine if chronic high level fructose metabolism leads to activation of stress pathways.

Obesity and fatty acid induced insulin resistance are thought to contribute to the progression of diabetes through the actions of the pathway (Yuan et al., Science, 293: 673-1677 (2001); Jonkers et al., Am. J. Med., 112: 275-280 (2002)). TNFα has long been recognized to induce lipolysis and insulin resistance, although the exact mechanism(s) by which this occurs has not been fully elucidated (Ventre et al., Diabetes, 46: 1526-1531 (1997); Uysal et al., Nature, 389: 610-614 (1997); Memon et al., Endocrinology, 132: 2246-2253 (1993)). Recently a relationship has been demonstrated between diet induced obesity and c-Jun-N-terminal kinase (JNK) activity (Hirosumi et al., Nature, 420:333-336 (2002)). JNK can be activated by either TNFα or by reactive oxygen intermediates (ROS) that are generated as a result of hyperglycemia-induced oxidative stress through a Rac→cytosoloic phospholipase A2→arachadonic acid pathway that generates ROS (Guha et al., J. Biol. Chem., 275:17728-17739 (2000)). The metabolism of arachadonic acid suggests a role for lipoxygenases in stress pathway signaling. Fructose fed animals exhibit reduced PPARA levels and a corresponding reduction of beta oxidation (Nagai et al., Am. J. Physiol. Endocrinol. Metab., 282: E1180-E1190 (2002)). As such, metabolism of xenobiotics, including lipoxygenase products, is likely impaired, which could result in their accumulation.

In order to address this hypothesis, the effects of lipoxygenase inhibitors on the metabolic and hepatic status of HFF rats were tested. Two structurally different LO inhibitors were examined because NDGA, which has previously demonstrated effects in this model (Scribner et al., Metabolism, 49: 1106-1110 (2000)), also exhibits numerous other biological effects. Interpretation of effects that might not be related to its LO inhibitor activity were thereby minimized. It is shown that fructose induces a hepatic stress response that mimics a portion of the TNFα acute phase response. This response occurs without overt hyperglycemia, obesity or significantly elevated fatty acids, suggesting that other metabolic triggers can induce inflammatory pathways that result in metabolic dysfunction.

Studies were performed in order to determine if the mechanism of lipid dysregulation in the high fructose diet is induced by stress response pathways. Animals were fed a high fructose diet for 14 days to establish hypertriglyceridemia and then were treated with lipoxygenase inhibitors for four days concurrent with the diet. At the end of drug treatment, the animals were divided into two groups and treated with LPS or a vehicle. Serum samples were taken pre-treatment and post-treatment and liver tissue was harvested at the end of study. Serum samples were tested for metabolic parameters and the tissue samples were tested for metabolic and stress pathway responses. Our results show that fructose fed rats have changes in the JNK pathway with correspondingly elevated AP-1 activity, consistent with an inflammatory response. Treatment with lipoxygenase inhibitors reversed the hypertriglyceridemia and also reduced AP-1 activation, suggesting that the basis for lipid dysregulation in this model is due to activation of inflammatory pathways in the liver.

Pharmaceutical Dosage Forms of the Present Invention

These pharmaceutical compositions will be formulated and dosed in a fashion consistent with good medical practice taking into account the method of administration, the scheduling of administration, and other factors known to practitioners. The “pharmaceutically effective amount” of each active agent for the purposes of the present invention is determined in view of such considerations. Those skilled in the art can readily determine empirically an appropriate “effective amount” of each active agent for a particular mammalian patient.

For medical use, the amount required of a compound or physiologically acceptable salt thereof (hereinafter referred to as the active ingredient) to achieve a therapeutic effect will, of course, vary both with the particular compound, the route of administration and the mammal under treatment. A suitable dose of a compound or physiologically acceptable salt thereof for a mammal is 0.1 μg-500 mg of base per kilogram bodyweight. In the case of oral administration, the dose may be in the range 0.5 mg to 500 mg of base per kilogram bodyweight, preferably about 1 mg to about 250 mg of base per kilogram bodyweight, most preferably about 5 mg to about 150 mg of base per kilogram bodyweight.

As used herein, the phrase “pharmaceutically acceptable” is intended to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “pharmaceutically acceptable carrier” is a pharmaceutically acceptable material, composition or vehicle, such as a liquid or solid filler, diluent, excipient, solvent or encapsulating material, involved in carrying or transporting the active agents of the inventive compositions from one organ, or portion of the body, to another organ, or portion of the body. Each carrier must be “acceptable” in the sense of being compatible with the other ingredients of the formulation and not injurious to the patient.

Some illustrative examples of materials which can serve as pharmaceutically-acceptable carriers include, but are not limited to, the following: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the inventive pharmaceutical compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, and the particular mode of administration. The amount of active ingredients that can be combined with a carrier material to produce a single dosage form will generally be that amount of each active ingredient that, together, produce the desired therapeutic effect. Generally, out of one hundred percent, this amount will range from about 0.01 percent to about ninety-nine percent of active ingredients, preferably from about 0.1 percent to about 90 percent, most preferably from about 1 percent to about 90 percent.

In general, the formulations are prepared by uniformly and intimately bringing into association the active ingredient with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of each active ingredient. The active ingredients of the inventive compositions may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents.

In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredients can also be in micro-encapsulated form, if appropriate, with one or more of the above-described excipients.

Liquid dosage forms for oral administration of the inventive compositions include pharmaceutically acceptable emulsions, microemulsions, solutions, suspensions, syrups and elixirs. In addition to the active ingredients, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredients, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

A second pharmaceutical dosage form of the present invention is one suitable for pulmonary administration via the buccal cavity. Preferably the composition is such that particles having a diameter of 0.5μ to 7μ, most preferably 1μ to 6μ, containing active ingredient, are delivered into the lungs of a patient. Such compositions are conveniently in the form of dry powders for administration from a powder inhalation device or self-propelling powder-dispensing containers, for example as a self-propelling aerosol composition in a sealed container; preferably the powders comprise particles containing active ingredient of which particles at least 98% by weight have a diameter greater than 0.5μ and at least 95% by number have a diameter less than 7μ. Most desirably at least 95% by weight of the particles have a diameter greater than 1μ and at least 90% by number of particles have a diameter less than 6μ.

The compositions in the form of dry powders preferably include a solid fine powder diluent such as sugar and are conveniently presented in a pierceable capsule, for example of gelatin.

Self-propelling compositions of the invention may be either powder-dispensing compositions or compositions dispensing the active ingredient in the form of droplets of a solution or suspension. Self-propelling powder-dispensing compositions include a liquid propellant having a boiling point of below 65° F. at atmospheric pressure. Generally the propellant may constitute 50 to 99.9% w/w of the composition whilst the active ingredient may constitute 0.1 to 20% w/w, for example about 2% w/w of the composition. The carrier in such compositions may include other constituents, in particular a liquid non-ionic or solid anionic surfactant, or a solid diluent (preferably having a particle size of the same order as of the particles of active ingredient) or both. The surfactant may constitute from 0.01 up to 20% w/w, though preferably it constitutes below 1% w/w of the composition.

Self-propelling compositions wherein the active ingredient is present in solution comprise an active ingredient, propellant and co-solvent, and advantageously an antioxidant stabilizer. The co-solvents may constitute 5 to 40% w/w of the composition, though preferably less than 20% w/w of the composition.

The following examples are illustrative, but not limiting, of the method and compositions of the present invention. Other suitable modifications and adaptations of the variety of conditions and parameters normally encountered in enzymatic production of chemicals and which are obvious to those skilled in the art are within the spirit and scope of the invention.

### EXAMPLES

### Example 1

### In Vivo Efficacy of 5-LO Inhibitors in an Animal Model of Hypertriglyceridemia and Hypertension

Animals and Treatments

Male Sprague-Dawley rats weighting 175-200 g were used in these studies. Rats weighting 175-200 g were used. Rats were first maintained on a chow diet for ˜1 wk and then were divided into four groups (7 or 8 animals in each group). Three groups were switched to a high-fructose diet (Harlan Teklad, Madison, Wis.) that provided 60% of total calories as fructose. The fourth group was maintained on normal chow to serve as an overall control group. After 14 days on the high-fructose diet, the degree of hypertriglyceridemia was evaluated by determining the total plasma TG levels. On day 0 of treatment, tail vein blood was collected for baseline measurements of serum TG, glucose, insulin, and FFA. The four groups of rats were then treated with either vehicle (0.5% carboxymethylcellulose), NDGA at a dose of 250 mg/k body wt, or BW 755c at a dose of 100 mg/kg body wt. All animals were dosed two times a day for 5 days (days 1-5), delivered by oral gavage. On day 5 of treatment (approximately 3 hours after the oral dose) vein blood was collected for measurements of serum TG, glucose, insulin, and FFA. During the treatment period, rats were maintained on their prescribed diets. Body weights were measured on day 0 and day 5.

Serum samples were used to measure TG and glucose concentrations by enzymatic calorimetric methods using Sigma Diagnostic kits (St. Louis, Mo.). Serum insulin concentrations were by RIA using a Linco Rat Insulin RIA kit (St. Charles, Mo.). FFA concentrations were measured using the nonesterified fatty acid (NEFA) C kit by the ACS-ACOD method following the instructions of the manufacturer.

Data was statistically analyzed using paired t-tests to determine significance.

Results

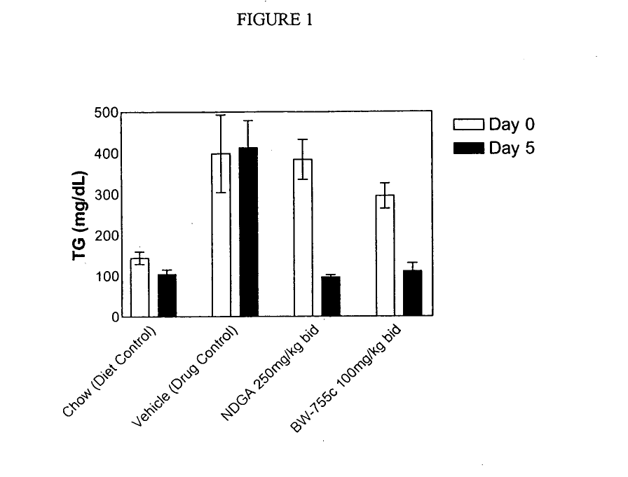

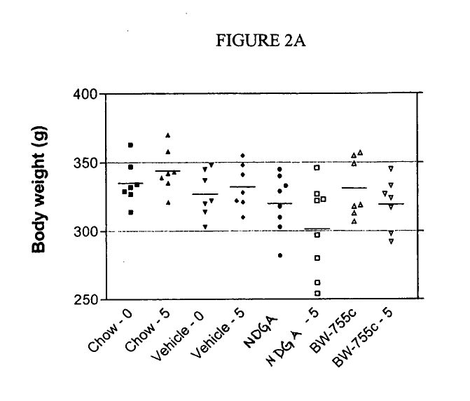

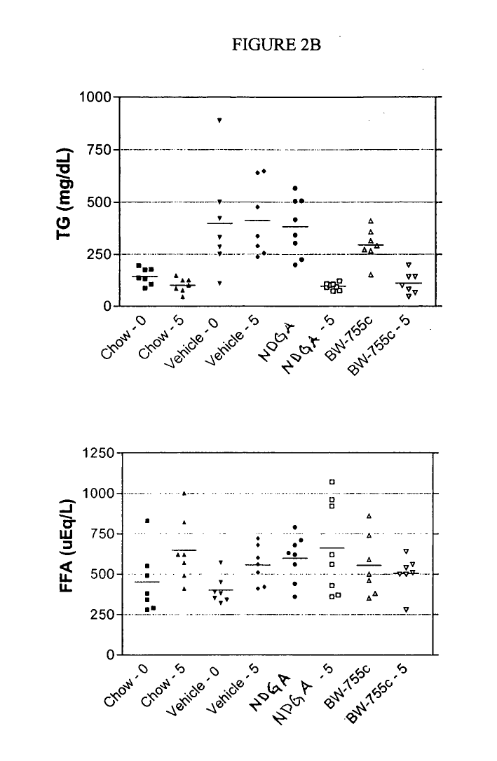

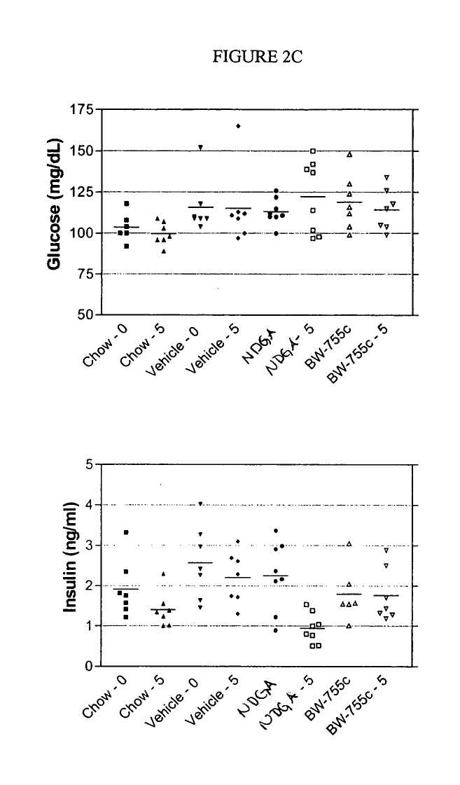

As expected, the high fructose diet induced an increase in serum triglycerides, which was significantly reduced in the NDGA (p=0.0004) and BW 755c (p=0.0002) groups (FIG. 1). During the course of treatment, the chow and vehicle groups exhibited a significant increase in serum free fatty acids (p=0.035 and p=0.045 respectively), and effect that was ameliorated in both the NDGA and BW 755c groups. In addition, NDGA (p=0.003) significantly lowered serum insulin levels without increasing serum glucose—an indication of increased insulin sensitivity. BW 755c had no effect on insulin or serum glucose levels, demonstrating a difference between the two compounds. No groups exhibited significant changes in body weight during the treatment period, though the animals in the NDGA group tended to gain less weight or overtly exhibited weight loss. The date for these groups is shown in scatter graphs in FIG. 2.

### EXAMPLE 2

Animals and Treatments

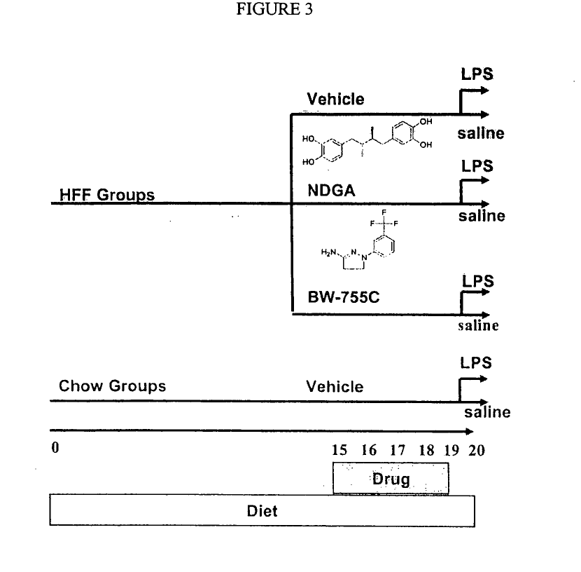

The treatment protocol is summarized in FIG. 3. Male rats were initially divided into HFF or chow (control) groups and maintained for 14 days on the prescribed diet. On day 15, the HFF groups were divided into three groups, vehicle, NDGA, and BW 755c (The chemical structures of NDGA and BW 755c are inset for illustration). On days 15-19 all groups were treated by oral gavage twice daily with either drug or vehicle. On days 15 and 19 serum was collected for analysis. On day 20, the groups were subdivided to receive either LPS or vehicle (saline). Post-LPS treatment, the animals were sacrificed and their livers were isolated for analysis.

Male Sprague-Dawley rats weighing approximately 180-200 g first maintained on a rat chow diet and then were divided into four groups, three of which were switched to a high-fructose diet (Harlan Teklad, Madison, Wis.) that provided 60% of total calories as fructose (Day 1). On day 15 of treatment, the rats were fasted for 4 hours and tail vein blood was collected for baseline measurements of serum TG, glucose, insulin and free fatty acids (FFA) as previously described (Gowri et al., Am. J. Hypertension, 12: 744-746 (1999)). The three groups of rats were then treated with either vehicle (0.5% carboxymethyl cellulose), NDGA (250 mg/kg BW) or BW 755c (100 mg/kg BW) BID for 4 days, delivered by oral gavage. The chow group (diet control) was treated with vehicle. During the treatment regimen the animals were maintained on high-fructose diet. Following 4 days of treatment, blood was collected from the tail vein 3 hours after last dose of vehicle, NDGA or BW 755c and serum samples were analyzed for TG, glucose, insulin, FFA, and total cholesterol as previously described (Gowri et al., 12: 744-746 (1999); Tercyak, J. Nutr. Biochem., 2:181-192 (1991); Kraemer et al., Endocrinology, 143: 801-806 (2002)). On day 20, four animals in each group were injected (iv) with 0.5 mg/kg BW Salmonella enterifdis endotoxin (lipopolysaccharide, LPS) or normal saline under light anaesthesia. After 2 hours, serum was collected for corticosterone measurement, as described previously (Kraemer et al., Endocrinology, 143: 801-806 (2002)), and then the animals were sacrificed and tissues removed, snap-frozen in liquid nitrogen and stored at −80° C. until analyzed. The local committee on animal care approved all animal protocols.

Measurement of Lipid Peroxidation by TBARS Assays

Membrane lipid peroxidation, as a measure of oxidative damage to lipids, was assessed by calorimetric determination of thiobarbituric acid-reactive substances (TBARS) on hepatic microsomal fractions as described (Azhar et al., J. Clin. Invest., 96: 1414-1424 (1995)). TBARS formation was determined under basal condition (endogenous) and in the presence of Fe^{e2+}/ADP/NADPH (enzymatic) and Fe ^{e2+}/ascorbate as pro-oxidants. Results are expressed as nmoles TBARS (malondialdehyde, MDA equivalent) formed/hr/mg protein.

Preparation of Hepatic Nuclear Extracts and Electrophoretic Mobility Shift Assays (EMSA)

Hepatic nuclear extracts were prepared according to the procedure described previously from this laboratory (Medicherla et al, Mech. Aging Dev., 122: 1169-1186 (2001)). For EMSAs, the double-stranded oligonucleotide probes were end-labeled using [γ-³²P] ATP and T₄ polynucleotide kinase and unincorporated radioactivity in each preparation was removed by Sephadex G-50 spin column chromatography. The double stranded sequences of the synthetic oligonucleotide containing AP-1 and SP-1 recognition sequence (the consensus sequences shown in bold) were as follows:

| | | |
|---|---|---|
| AP-1 (TRE)-- | 5′-CGCTTGATGAGTCAGC | (SEQ ID NO: 1) |
| | CGGAA-3′ | |
| | 3′-GCGAACTACTCAGTCG | (SEQ ID NO: 2) |
| | GCCTT-5′ | |
| SP-1-- | 5′-ATTCGATCGGGGCGGG | (SEQ ID NO: 3) |
| | GCGAGC-3′ | |
| | 3′-TAAGCTAGCCCCGCCC | (SEQ ID NO: 4) |
| | CGCTCG-5′ | |

Each reaction mixture (20 μl) for AP-1 contained: 15 mM HEPES-NaOH (pH 7.9), 3 mM Tris-HCl (pH 7.9), 60 mM KCl, 0.5 mM EDTA, 1 mM MgCl₂, 100 μg/ml poly (dI-dC).poly (dI-dC), 0.5 mM DTT, 1% NP-40, 10% glycerol ³²P-labelled double-stranded oligonucleotide probe (˜100,000 DPM) and 4.0-8.0 μg nuclear protein extract; for SP-1 (20 μl): 50 mM Tris-HCl (pH 7.9), 100 mM KCl, 12.5 mM MgCl₂, 1 mM DTT, 100 μg/ml poly (dI-dC).poly (dI-dC), 1 mM DTT, 1% NP-40, 10% glycerol, ³²P-labelled double-stranded oligonucleotide probe (˜100,000 DPM) and 4.0-8.0 μg nuclear protein extract. The ³²P-oligonucleotide-nuclear protein complexes formed were separated from free oligonucleotide by polyacrylamide gel electrophoresis. Following electrophoresis, the gels were dried and exposed to Kodak X-OMAT film for appropriate time (≦72 h), and were then scanned and the appropriate bands quantified by densitometry. The results are expressed as arbitrary units/10 μg nuclear protein extract.

Western Blot Analysis of Total and Phosphorylated Forms of ERKs, p38MAPK and JNKs

Liver samples (˜200 mg) were homogenized using a Potter-Elvehjum homogenizer in 3 volumes of detergent containing lysis buffer [20 mM HEPES, pH 7.4, 1% Triton X-100 (v/v), 150 mM NaCl, 1 mM EDTA, 1 mM EGTA, 20 mM NaF, 20 mM β-glycerophosphate, 10 mM sodium pyrophosphate, 1 mM sodium vanadate, 10 mM okadaic acid, 1 mM dithiothreitol, 10 μg/ml aprotinin, 10 μg/ml leupeptin, 10 μg/ml pepstatin A, 0.5 mM 4-(2-aminoethyl)benzylsulfonyl fluoride (AEBSF, Roche Molecular Biochemicals), 10 μM E-64 and 50 μM Bestatin] and incubated for 30 min at 4° C. on an orbital shaker for complete lysis. The lysates were cleared by centrifugation at 15,000×g for 10 min, the protein concentration of each solubilized lysate was determined and samples stored frozen until analyzed.

Samples containing an equal amount of protein (80 μg) were fractionated by SDS-polyacrylamide gel electrophoresis (10% polyacrylamide gel with 4% stacking gel) and transferred to polyvinyllidene difluoride membrane (Immobilon™, Millipore Corp., Bedford, Mass.). After transfer, the membrane was washed in TBS containing 0.1% Tween-20 (TTBS) and incubated in blocking buffer (TTBS containing 5% non-fat dry milk) for 90 min at room temperature followed by overnight incubation at 4° C. with primary antibody diluted in blocking buffer. Subsequently, the membrane was washed in TTBS and incubated for 2 hr with horseradish peroxidase conjugated secondary antibody in blocking buffer. The immunoreactive bands were then visualized using LumiGLO Chemiluminescent Detection System (KPL Laboratories) followed by exposure to X-ray film (15-35 minutes) and quantified by Fluor-S-Multilmager scanning densitometry system (Bio-Rad). Polyclonal antibodies against total ERKs, JNKs/SAPKs and p38 MAPK were purchased from Cell Signaling Technology (Beverly, Mass.). Phospho-specific antibodies against phosphorylation of p38 MAPK ((Thr¹⁸⁰/Tyr¹⁸²) and ERKS (Thr²⁰²/Tyr²⁰⁴) were also supplied by Cell Signaling Technology. Phospho-JNKs/Phospho-SAPKs (Thr¹⁸³/Tyr¹⁸⁵) antibody was obtained from Santa Cruz Biotechnology, Inc. (Santa Cruz, Calif.).

Statistical Analysis

Statistical analysis were performed by either paired or unpaired t-test using GraphPad Prism version 3.00 for Windows, GraphPad Software, San Diego Calif. USA. A difference between groups was considered significant if P was less than 0.05. Normalized Western Blot data were obtained as a ratio of the units measured for phosphorylated protein divided by the units measured for total protein. Statistical analyses were performed on the normalized data.

Results

Lipoxygenase Inhibitors Reduce Hypertriglyceridemia

As shown Table 1, the high fructose diet induced dramatic hypertriglyceridemia and significant increased serum glucose and total cholesterol. Importantly, at this stage of treatment, these animals are not obese and do not exhibit elevated FFA and their blood glucose levels are not dangerously high. After four days of treatment, both of the LO inhibitors reduced serum TG to chow-fed control levels, completely reversing the effects of the HFF diet (Table 2). During the treatment phase, the chow group exhibited a small, but significant decrease in serum TG and a statistically significant increase in serum FFA. The corresponding vehicle HFF group also exhibited a small, but significant increase in FFA. The effect of lowered serum TG and elevated FFA may have been due to generalized animal stress as a result of twice daily oral gavage treatment and was not considered a specific effect of the dietary treatment. Neither group receiving the LO inhibitors exhibited this increase in serum FFA. NDGA significantly decreased fasting serum insulin without significantly altering fasting blood glucose and significantly reduced serum total cholesterol. Neither of these effects was observed with BW 755c suggesting that they are NDGA-specific and presumably unrelated to LO activity.

TABLE 1
Baseline metabolic parameters of fructose fed animals

| | Chow - Day 15 | HFF Diet - Day 15 |
|---|---|---|
| Body Weight (g) | 335 ± 6 | 326 + 4 |
| Glucose (mg/dL) | 94.6 ± 10 | 116 ± 3 (a′ |
| Insulin (ng/mL) | 1.9 ± 0.3 | 2.1 + 0.2 |
| TG (mg/dL) | 145 ± 15 | 360.9 ± 35.7 (b) |
| FFA (gEq/L) | 451 ± 74 | 521.4 ± 35 |
| Cholesterol (mg/dL) | 87.8 ± 4 | 111 ± 3 ^{(c)} |

Data presented as mean ± S.E. (Chow n = 7-8; Fructose n = 22-24). All statistics analyses were unpaired t-test comparing chow vs HFF diets.
(a) p < 0.007;
(b) p < 0.002;
^{(c)} p < 0.0003

TABLE 2
Effect of Lipoxygenase inhibitors on serum metabolic components

| | Glucose | | Insulin | | TG | | FFA | | Cholesterol | |
|---|---|---|---|---|---|---|---|---|---|---|
| | (mg/dL) | | (ng/mL) | | (mg/dL) | | (μEq/L) | | (mg/dL) | |
| Group | Day 15 | Day 19 | Day 15 | Day 19 | Day 15 | Day 19 | Day 15 | Day 19 | Day 15 | Day 19 |
| Chow | 95 ± 9 | 100 ± 3 | 1.9 ± 0.3 | 1.3 ± 0.2 | 144 ± 15 | 104 ± 12(²) | 451 ± 74 | 647 ± 76(⁵) | 87.8 ± 4 | 82.3 ± 3 |
| HFF | 116 ± 6 | 115 ± 9 | 2.6 ± 0.3 | 2.2 ± 0.2 | 399 ± 94 | 413 ± 67 | 400 ± 32 | 557 ± 45⁽⁵⁾ | 106.9 ± 7 | 103.3 ± 5 |
| HFF + | 113 ± 3 | 122 ± 8 | 2.3 ± 0.3 | 0.9 ± 0.1(′) | 384 ± 48 | 97 ± 6(s) | 599 ± 51 | 611 ± 101 | 118.6 ± 3 | 98.3 ± 4(⁷) |
| N DGA | | | | | | | | | | |
| HFF + | 119 ± 7 | 114 ± 7 | 1.6 ± 0.3 | 1.7 ± 0.3 | 296 ± 31 | 112 ± 20(⁴) | 554 ± 71 | 504 ± 42 | 107.6 ± 3 | 104.5 ± 2 |
| BW755 | | | | | | | | | | |

Data presented as the mean t S.E. (n = 7-8).
All statistical analyses were paired t-tests comparing Day 15 vs Day 19 of each group.
(1) p < 0.003; (2) p < 0.035; (3) p < 0.004; (4) p < 0.002; (5) 24 < 0.035; (6) p < 0.045; (7) p < 0.01

Effects of Fructose Feeding on Hepatic Lipid Metabolism

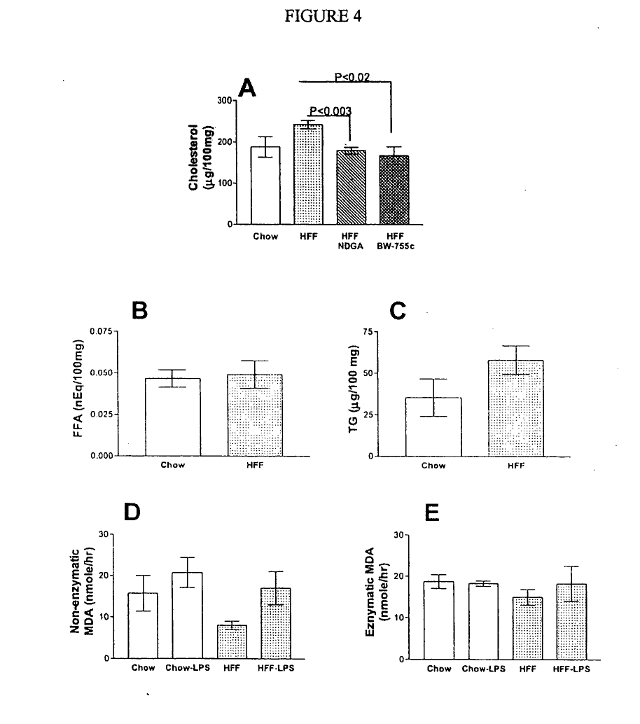

After the treatment protocol, we examined the hepatic lipid composition in the saline-treated animals to determine the effect of LO inhibitors on hepatic lipid metabolism. FIGS. 4A-4E show these results. Chow diet=clear bars; HFF diet=dotted bars; HFF diet+NDGA treatment=stripped bars; HFF+BW 755c treatment=hatched bars. All data presented as mean±SE, n=4 per group. As seen in FIG. 4A, the total cholesterol content of HFF animals was elevated compared to chow controls. The total hepatic cholesterol of chow and HFF animals is expressed as micrograms cholesterol per 100 milligrams of tissue. The LO inhibitors reduced total cholesterol, an effect consistent with restoration of normal hepatic VLDL metabolism. In contrast to serum lipids, liver TG content and liver FFA were not significantly elevated in the HFF animals (FIGS. 4B,C). In FIG. 4B, hepatic free fatty acid content of chow and HFF animals is expressed in nanoequivalent units per 100 milligrams of tissue. FIG. 4C shows the hepatic triglyceride content of chow and HFF animals expressed in micrograms per 100 milligrams of tissue. This suggests that these HFF animals did not exhibit impaired hepatic TG or FFA secretion during the treatment period. Finally we measured the extent of membrane lipid peroxidation, which increases under conditions of oxidative stress, and products of which are known to activate JNK and hence AP-1 (Uchida et al, J. Biol. Chem., 274: 2234-2242 (1999)). Liver microsomal fractions from HFF animals demonstrated similar susceptibility to lipid oxidation in response to enzymatic prooxidants and a reduced susceptibility to non-enzymatic pro-oxidants (FIGS. 4D,E). FIG. 4D shows the lipid peroxidation of chow and HFF hepatic microsomes by nonenzymatic TBARS assay. FIG. 4E shows the lipid peroxidation of chow and HFF hepatic microsomes by enzymatic TBARS assay. Animals from all groups that were treated with LPS exhibited similar sensitivity to microsomal lipid peroxidation including negating the non-specific anti-oxidant effects of the HFF diet.

Fructose Feeding Induces a Hepatic Stress Response

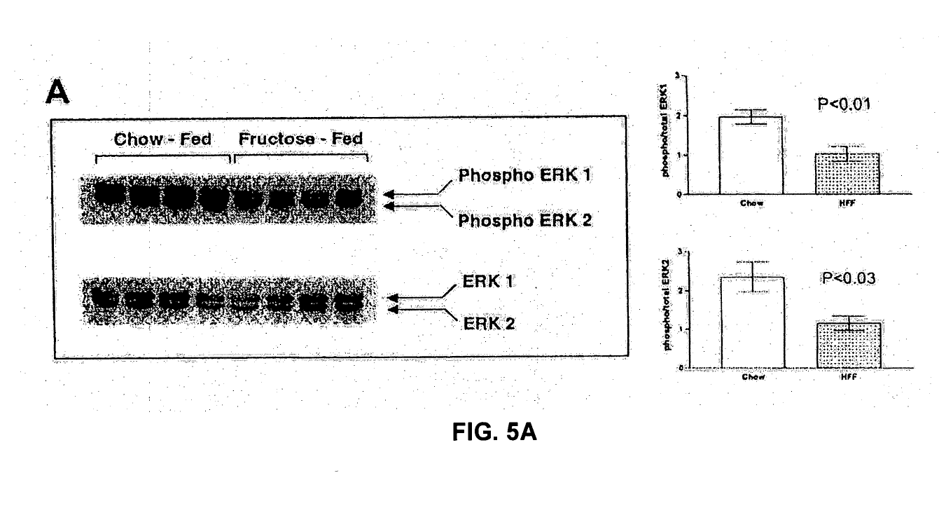

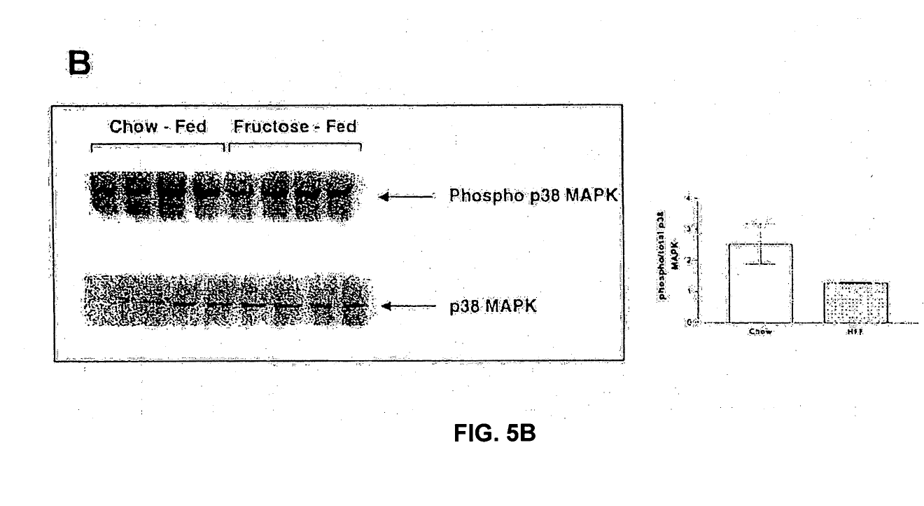

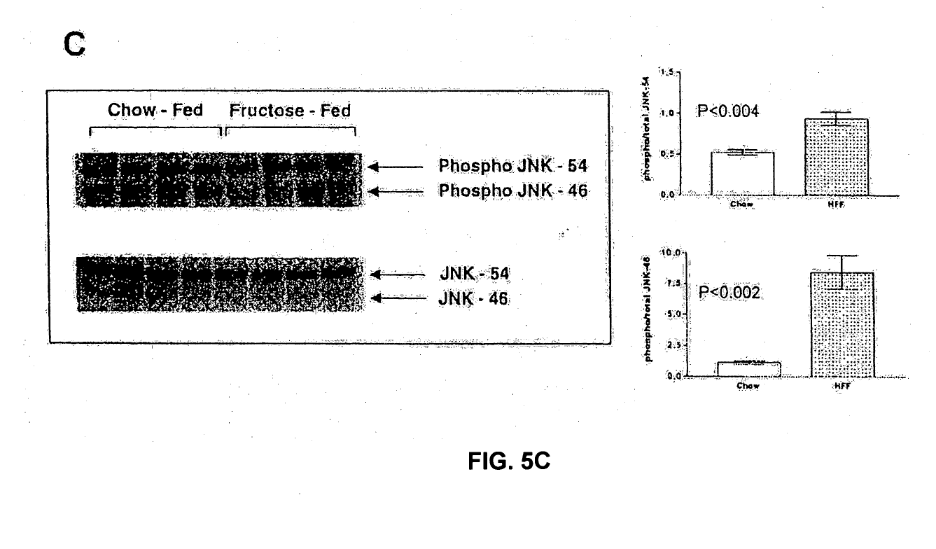

Due to the similarity between metabolic effects of high fructose diets and inflammatory acute responses on serum lipids, we examined the effects of the HFF diet on certain elements of the stress pathway that have been associated with insulin resistance. As shown in FIG. 5, HFF animals exhibited significant reduction in the total protein of JNK p54 and p46, particularly p46. FIGS. 5A-5C show western blot analyses of kinase activity. Chow diet=clear bars; HFF diet=dotted bars. The bar graphs indicate the ratio of phosphorylated kinase to total kinase protein of the individual samples expressed as the mean±SE n=4 per group. FIG. 5A shows total and phosphorylated ERKI and ERKI western blots for chow and HFF animals. FIG. 5B shows total and phosphorylated p38 MAP kinase western blots for chow and HFF animals. FIG. 5C shows total and phosphorylated JNK-46 and JNK-54 western blots for chow and HFF animals. Despite the reduction of total protein, both isoforms of JNK exhibited significant phosphorylation, indicating that these pathways are activated upon chronic fructose metabolism. The HFF animals did not exhibit significant changes to p38 MAPK, either total protein or phosphorylation state. ERKI/2 total protein levels were comparable between groups, but the phosphorylation state of these kinases was reduced by approximately 50% in HFF animals.

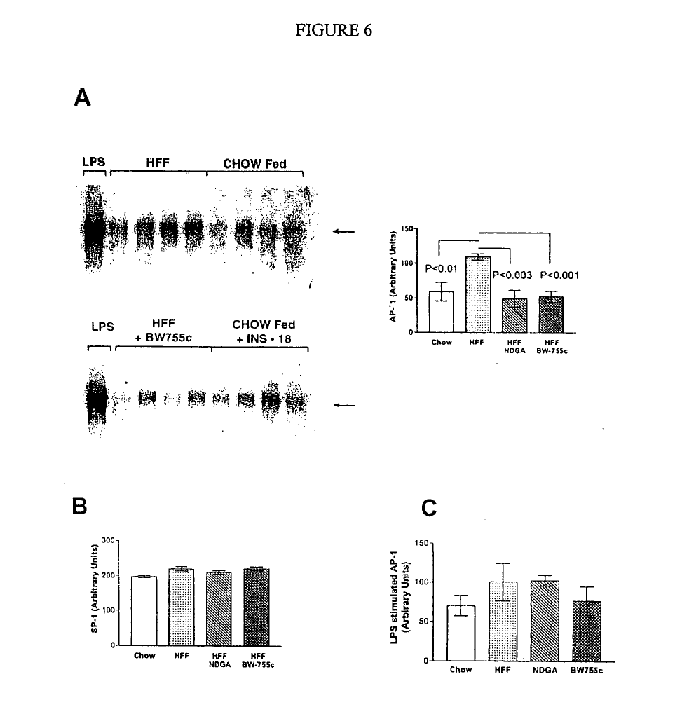

To corroborate the observation of JNK pathway activation in HFF animals, we examined the DNA binding activity of AP-1. FIGS. 6A-6C show quantified EMSA analysis of hepatic AP-1 and SP-1. Chow diet=clear bars; HFF diet=dotted bars; HFF diet+NDGA treatment=stripped bars; HFF+BW 755c treatment=hatched bars. All data presented as mean±SE, n=4 per group. FIG. 6B shows the densitometric intensity of a 3-day exposure of SP-1 for saline-treated groups. FIG. 6C shows the densitometric intensity of an overnight exposure of AP-1 for LPS-treated groups. As seen in FIG. 6A, the HFF diet increased the intensity of the AP-I EMSA band by 86% compared to the chow diet. 3-day exposure of AP-1 for saline-treated groups. The densitometric intensity of each group is expressed as arbitrary units/10 Rg nuclear protein extract and plotted on a graph to the right side of the figure. Treatment with both compounds significantly reduced AP-I band intensity as compared with the HFF diet to levels that were nearly identical to the chow group. As a control, we compared the diet-induced changes of AP-I activity to that of another transcription factor, SP-I (FIG. 6B). Neither the fructose diet, nor the LO inhibitors affected activity of SP-I relative to the chow diet. The reduction of AP-I activity in the LO inhibitor groups was due the overall status of the liver and not an acute drug effect because the livers were harvested a full day after the last drug treatment, thus allowing drug clearance prior to AP-I measurement. In support of this conclusion is the observation that all groups challenged with LPS exhibited a robust response to AP-1 (FIG. 6C).

Hepatic Lipid Metabolism Dysregulation in the HFF Model is not Due to Corticosterone

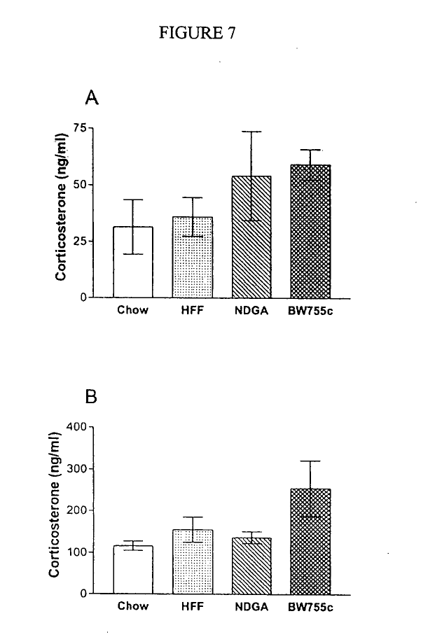

In rats, fructose feeding increases serum levels of corticosterone, a glucocorticoid that, in part, regulates hepatic activity of phosphohydrolase and thus influences hepatic TG synthesis (Knox et al., Biochem. J, 180: 441-443 (1979); McIntosh et al., Proc. Soc. Exp. Med., 221: 198-206 (1999); Brindley et al., Biochem. J, 180: 195-199 (1979)). Therefore it is possible that one effect of LO inhibitors may be to suppress endogenous corticosterone production and thus indirectly influence hepatic lipogenesis. In order to address this question we measured serum corticosterone in the groups after drug treatment. FIGS. 7A-7B show serum corticosterone measurement. Chow diet=clear bars; HFF diet=dotted bars; HFF diet+NDGA treatment=stripped bars; HFF diet+BW 755c treatment=hatched bars. All data presented as mean±SE, n=4 per group.

All animals exhibited elevated levels of serum corticosterone, which most likely was due to overall animal stress precipitated by repeated animal handling. FIG. 7A shows serum from day 20, saline treated groups. Animals receiving NDGA and BW 755c exhibited slightly elevated serum levels of corticosterone, though these levels were not significantly higher than those for fructose animals. FIG. 7B shows the serum from Day 20 for LPS treated groups. LPS treatment resulted in a robust corticosterone response in all groups, with a slightly elevated, but not statistically significant, response in the BW 755c group. Collectively, the fructose-induced effects on hepatic TG production do not appear to be related to the adrenal stress response or general stress of the animals.

These studies demonstrate that a high fructose diet induces a hepatic response through the JNK/AP-1 pathway, which is similar to that observed for the inflammatory cytokine TNFα. In addition to activation of the JNK pathway, it appears that the ERKI/2 pathway is suppressed suggesting reduced mitogenic kinase activity in the presence of increased stress kinase activity. We demonstrated that two compounds, both lipoxygenase inhibitors, reduced activation of AP-1 and reduced serum hypertriglyceridemia, which is the metabolic hallmark of this model. These results suggest a link between the hepatic inflammatory status and lipid dysregulation.

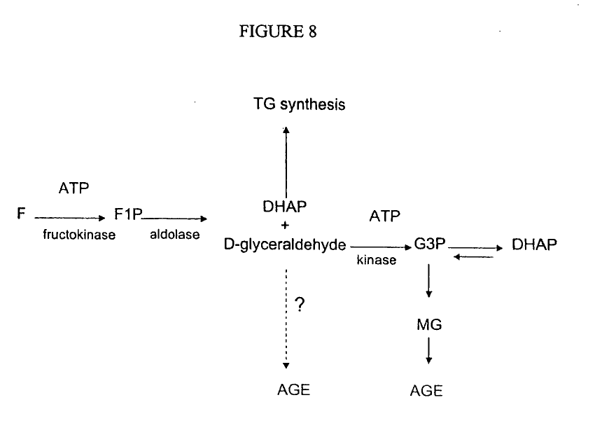

The mechanism by which these LO inhibitors reverse the stress response is unresolved. One mechanism by which these drugs may work is through inhibition of ROS generation and thus directly disrupt the JNK pathway, similar to that seen during hyperglycaemic stress responses (Guha et al., J. Biol. Chem., 275:17728-17739 (2000)) though this appears unlikely since the HFF TBARS values (a crude measure of lipid peroxidation and oxidative damage to membranes) were unremarkable relative to chow controls. Moreover, β-oxidation appears to be down regulated due to an ample energy supply in the form of fructose. Therefore this excludes lipid peroxidation as the primary cause of the AP-1 activation in the HFF model. Alternatively, hepatic metabolism of fructose may generate stress activating molecules directly. Fructose is metabolized in the liver to yield dihydroxyacetone phosphate (DHAP) and D-glyceraldehyde, which can be phosphorylated to glyceraldehyde-3-phosphate (G3P). DHAP and G3P are glycolytic intermediates and intermediates in TG synthesis. Because fructose metabolism is not regulated like glucose, it is theoretically possible that excess consumption of this sugar could lead to elevated levels of DHAP and G3P if they were not utilized (for example in the case of rested rats). We propose a theory that accumulation of methylglyoxal and/or D-glyceraldehyde could provide substrate for glyceraldehyde derived advanced glycation end products (glycer-AGE) as shown in FIG. 8. The hepatic metabolism of fructose is depicted in this Figure. F=fructose; FIP=fructose-1-phosphate; DHAP=dihydroxyacetonephosphate; TG=triglyceride; G3P=glyceraldehyde-3-phosphate; MG=methylglyoxal AGE=advanced glycation end product. Fructose is metabolized in the liver to FIP by fructokinase and an aldolase to yield DHAP and D-glyceraldehyde. D-glyceraldehyde can be phosphorlyated to yield the glycolytic intermediate G3P. G3P can either be metabolized or can isomerize to yield DHAP. Alternatively G3P may form AGEs through MG as a Hepatic effect of high dietary fructose fragmentation intermediate. Theoretically, D-glyceraldehyde may directly conjugate with cellular proteins to yield AGEs.

Methylglyoxal has been associated with NF-kB activation and diabetic complications (Hammes et al., Nature Medicine, 9: 294-299 (2003)), while D-glyceraldehyde has demonstrated increased transcription activation of AP-1 in endothelial cells (Okamoto et al., FASEB J., 16: 1928-1930 (2002)). As such, LO inhibitors could inhibit JNK pathway activation from these aldehyde intermediates (Woo et al., J. Biol. Chem., 275: 32357-32362 (2000)). This mechanism may account for the observation that rats fed a HFF diet in conjunction with exercise do not develop hypertriglyceridemia, because these glycolytic intermediates may be shuttled through glycolysis rather than accumulating and/or being utilized in alternative metabolic or chemical pathways.

Most data in the literature focus on inflammatory effects that are caused by or exacerbated by hyperglyceridemia, obesity, or elevated free fatty acids. An evolving hypothesis is that metabolic disease and inflammation create a progressive cycle leading to disease progression and metabolic and cardiovascular complications (Yuan et al., Science, 293: 673-1677 (2001); Jonkers et al., Am. J. Med., 112: 275-280 (2002)). Our data agree with this hypothesis and we propose that low-level inflammation may occur prior to the onset of overt metabolic disease, thus allowing one potential entry point into the cycle. Assuming this to be true, these data suggests that leukotriene inhibitors may be useful drugs to treat certain individuals with metabolic disease. These data further support the concept that diabetics and other groups at risk should evaluate their consumption of high levels of dietary fructose in light of possible dyslipidemia.

Having now fully described this invention, it will be understood to those of ordinary skill in the art that the methods of the present invention can be carried out with a wide and equivalent range of conditions, formulations, and other parameters without departing from the scope of the invention or any embodiments thereof. All patents and publications cited herein are hereby fully incorporated by reference in their entirety.

What is claimed is:

1. A method for treating elevated serum triglycerides or hypertension comprising administering to a human subject with elevated serum triglycerides or hypertension an effective amount of pharmaceutical composition comprising a 5-lipoxygenase inhibitor, said effective amount being sufficient to reduce said elevated serum triglycerides or hypertension, wherein said 5-lipoxygenase inhibitor is not NDGA or curcumin.

2. The method of claim 1, wherein elevated serum triglycerides is treated.

3. The method of claim 1, wherein hypertension is treated.

4. The method of claim 1, wherein the pharmaceutical composition is an oral dosage form.

5. The method of claim 1, wherein said 5-lipoxygenase inhibitor is selected from the group consisting of an acetohydroxamic acid derivative, a phenyl pyrazoline derivative, a 2-(12-hydroxydodeca-5,10-diynyl)-3,5,6-trimethyl-1,4-benzoquinone derivative, and a 3-[1-(4-chlorobenzyl)-3-t-butyl-thio-5-isopropylindol-2-yl]-2,2-dimethyl propanoic acid derivative.

6. The method of claim 5, wherein said 5-lipoxygenase inhibitor is an acetohydroxamic acid derivative

7. The method of claim 6, wherein said acetohydroxamic acid derivative is N-(3-phenoxycinnamyl)acetohydroxamic acid (BW 4AC).

8. The method of claim 5, wherein said 5-lipoxygenase inhibitor is a phenyl pyrazoline derivative.

9. The method of claim 8, wherein said phenyl pyrazoline derivative is 4,5-dihydro-1-(3-(trifluoromethyl)phenyl)-1H-pyrazol-3-amine (BW 755c).

10. The method of claim 5, wherein said 5-lipoxygenase inhibitor is a 2-(12-hydroxydodeca-5,10-diynyl)-3,5,6-trimethyl-1,4-benzoquinone derivative.

11. The method of claim 10, wherein said derivative is 2-(12-hydroxydodeca-5,10-diynyl)-3,5,6-trimethyl-1,4-benzoquinone (AA861).

12. The method of claim 5, wherein said 5-lipoxygenase inhibitor is a 3-[1-(4-chlorobenzyl)-3-t-butyl-thio-5-isopropylindol-2-yl]-2,2-dimethyl propanoic acid derivative.

13. The method of claim 12, wherein said derivative is 3-[1-(4-chlorobenzyl)-3-t-butyl-thio-5-isopropylindol-2-yl]-2,2-dimethyl propanoic acid (MK886).

14. The method of claim 1, wherein said effective amount of said 5-lipoxygenase inhibitor is between 0.1 μg and 500 mg per kilogram of body weight.

15. The method of claim 14, wherein said effective amount of said 5-lipoxygenase inhibitor is between 0.5 mg to 500 mg per kilogram of body weight.

16. The method according to claim 1, further comprising administering a second compound selected from the group consisting of anti-diabetic compounds, lipid-lowering medications and anti-hyptertensive compounds.

17. The method according to claim 14, wherein the 5-lipoxygenase inhibitor and said second compound are administered concurrently.
